(19) United States
(12) Patent Application Publication
(10) Pub. No.: US 2005/0033336 A1
(43) Pub. Date: Feb. 10, 2005

(54) Safety surgeon knife structure

(76) Inventor: Chang-Ming Yang, Junan Jen (TW)

Correspondence Address: DENNISON, SCHULTZ & DOUGHERTY, 612 Crystal Aquare 4, 1745 Jefferson Davis Highway, Arlington, VA, 22202-3417, US

(21) Appl. No.: 10/634,957

(22) Filed: Aug. 6, 2003

(51) Int. Cl. A61B 17/32

(52) U.S. Cl. 606/167

(57) ABSTRACT

A safety surgeon knife structure comprising mainly a knife handle, a knife seat, a spring and a protective coat, there settles an allocation portion on said knife handle, and there settles a sliding manger for the insertion of the sliding block settled on the knife seat on the bottom of the allocation portion, and the holes is in connection with the allocation portion, and the spring on the rear end of the knife seat is in connection with the rear end wall facet of the allocation portion, the surgeon knife could be placed within the allocation manger by means of controlling the sliding movement of the sliding block of the knife seat in the sliding manger which fixes the knife seat within the rear end of the knife handle.

### BACKGROUND OF THE INVENTION

1. Field of the Invention

This invention relates to a safety surgeon knife structure, especially refer to a safety surgeon knife structure of which it could prevent the damage hurt by out-through of the edge portion of the surgeon knife.

2. Description of the Prior Art

Accordingly, the general conventional surgeon knife is to put a putting seat on the front end of a knife handle, when need to use it, it clamped the knife of the surgeon knife with a needle holder, next the putting seat is penetrating into the holding hole settled on the knife of the surgeon knife which fixed the razor blade of the surgeon knife on the knife handle; however, since the nurses should first fix the knife of the surgeon knife before taking it to the surgeon doctor on surgeon operation, so it is easily happen that the nurses or the doctors are damaged by the knife of the surgeon knife accidentally to cause danger or even infection.

Thus it is easily to see that there are many drawbacks for the above-mentioned object which requires to be bettered.

The inventor of this invention, due to many drawbacks derived from the above-mentioned conventional surgeon knife, thought to better it and studied for many years and finally invent the safety surgeon knife structure of this invention.

### SUMMARY OF THE INVENTION

The object of this invention is to provide a safety surgeon knife structure of which it could envelop the razor blade of the surgeon knife when not in usage.

The safety surgeon structure which could achieve the above-mentioned object of invention comprises:
A knife handle, there settles an allocation portion on said knife handle, on the bottom of said allocation portion there settles a sliding manger, and there settles a manger on both ends of the sliding manger;
A knife seat, there settles a surgeon knife on the front end of said knife seat, and there settles a sliding block being put inside the sliding manger of the knife seat; and
A spring;
Said knife seat is placed in the allocation portion of the knife handle, wherein the sliding block on the bottom of the knife seat is allocated within the sliding manger of the knife handle, and one end of said spring is connecting to the rear end of the knife seat, another end of said spring is connected with the wall on the rear end of the allocation portion of the knife handle.

### BRIEF DESCRIPTION OF THE DRAWINGS

The drawings disclose an illustrative embodiment of the present invention which serves to exemplify the various advantages and objects hereof, and are as follows:

FIG. 1 is the solid composition view of the safety surgeon knife structure of this invention;

FIG. 2 is the side cross-sectional view of the safety surgeon knife structure of this invention;

FIG. 2A s the partial cross-sectional top view of the safety surgeon knife structure of this invention;

FIG. 3 is the solid appearance view of the safety surgeon knife structure of this invention;

FIG. 4 is the usage status illustrative view of the safety surgeon knife structure of this invention;

FIG. 5 is the lateral cross-sectional view of the first embodiment of the safety surgeon knife structure of this invention;

FIG. 6 is the lateral cross-sectional view of the second embodiment of the safety surgeon knife structure of this invention;

FIG. 6A is the partial solid cross-sectional view of the second embodiment of the safety surgeon knife structure of this invention (knife handle 1);

FIG. 6B is the partial solid cross-sectional view of the second embodiment of the safety surgeon knife structure of this invention (2);

FIG. 7 is the solid appearance view of the third embodiment of the safety surgeon knife structure of this invention;

FIG. 8 is the solid appearance view of the fourth embodiment of the safety surgeon knife structure of this invention;

FIG. 9 is the partial solid cross-sectional view of the fifth embodiment of the safety surgeon knife structure of this invention;

FIG. 9A is the partial solid cross-sectional view of the sixth embodiment of the safety surgeon knife structure of this invention;

FIG. 10 is the solid appearance view of the seventh embodiment of the safety surgeon knife structure of this invention;

FIG. 10A is the solid appearance view of the eighth ninth embodiment of the safety surgeon knife structure of this invention;

FIG. 10B is the solid appearance view of the ninth embodiment of the safety surgeon knife structure of this invention;

FIG. 11 is the solid appearance view of the tenth embodiment of the safety surgeon knife structure of this invention;

FIG. 11A is the solid envelop view of the protective envelop of the tenth embodiment of the safety surgeon knife structure of this invention;

FIG. 11B is the illustrative view of the usage status of another protective envelop of the tenth embodiment of the safety surgeon knife structure of this invention;

FIG. 12 is the illustrative view of the usage status of said put seat (1);

FIG. 12A is the illustrative view of the usage status of said put seat (2);

FIG. 12B is the illustrative view of the usage status of said put seat (3);

FIG. 13 is the solid appearance view of another embodiment of said put seat of this invention;

FIG. 14 is the solid appearance view of the knife handle eleventh embodiment of said put seat of this invention;

FIG. 14A is the side cross-sectional view of the knife handle eleventh embodiment of said put seat of this invention;

FIG. 14B is the partial top view of the knife handle eleventh embodiment of said put seat of this invention;

FIG. 14C is the partial solid cross-sectional view of the knife handle eleventh embodiment of said put seat of this invention;

FIG. 15 is the solid appearance view of the knife handle 12th embodiment of said put seat of this invention;

FIG. 16 is the side cross-sectional view of the knife handle 13th embodiment of said put seat of this invention;

FIG. 17 is the side cross-sectional view of the knife handle 14th embodiment of said put seat of this invention;

### DETAILED DESCRIPTION OF THE PREFERRED EMBODIMENT

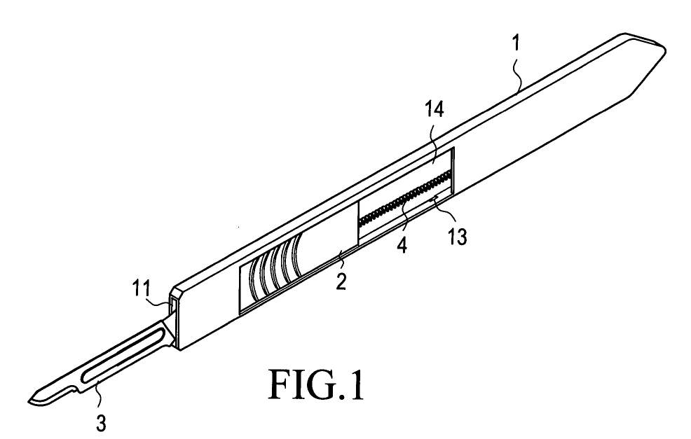

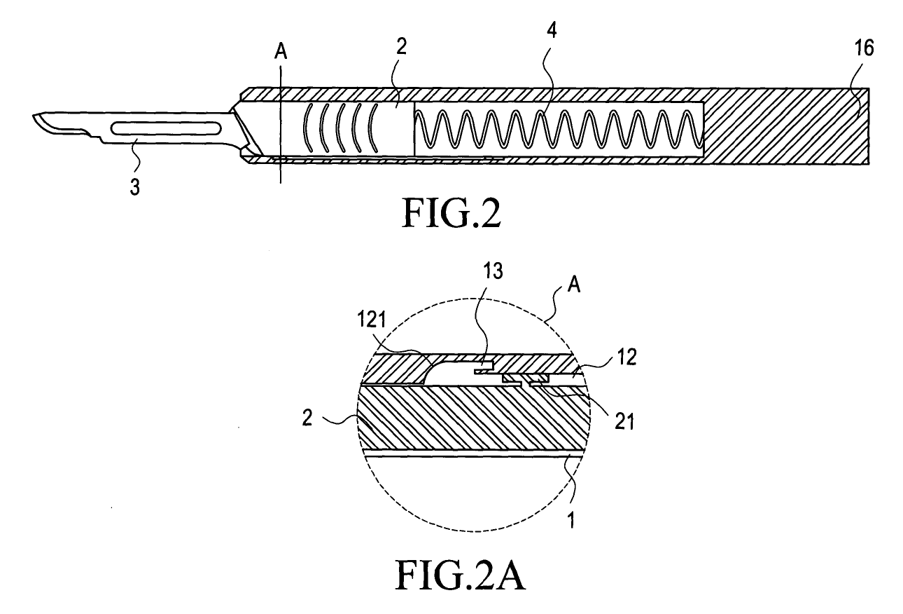

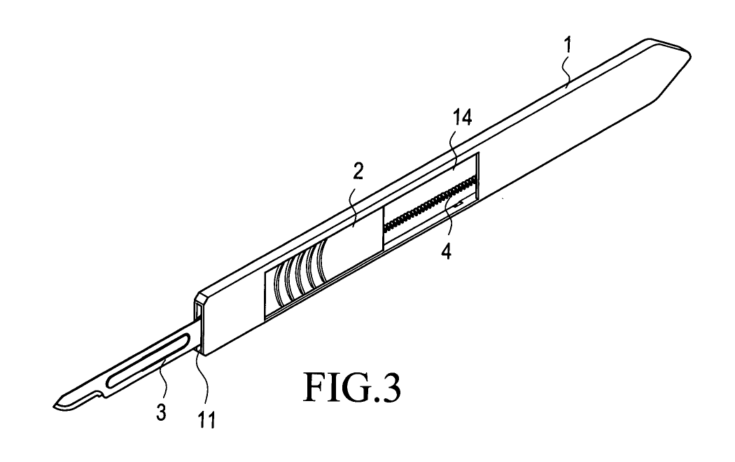

Please refer to FIG. 1, FIG. 2, FIG. 2A and FIG. 3, the safety surgeon knife structure provided by this invention comprises mainly:
A knife handle 1, wherein there settles an allocation portion knife handle 1 concave settled on the front end of said knife handle 1, there settles a sliding manger knife handle 12 on the bottom of said allocation portion knife handle 1, and on the front end and the rear end of the sliding manger 12 there settles a manger 13 in parallel with the sliding manger 12, and there each forms a guiding angel 12 on the lateral wall on the front end and the rear end of the sliding manger 12, wherein one lateral of knife handle 1 there settles a penetrating holes 14 which penetrates with the allocation portion knife handle 1;
A knife seat 2, there settles a surgeon razor blade 3 on the front end of said knife seat 2, and there settles a sliding block on the bottom of knife seat 2 putting insert the sliding manger 12 of knife handle 1;
A spring 4;
Said knife seat 2 is settled within the allocation portion knife handle 1, whereas the sliding block 2 on the bottom of the knife seat 2 is allocated within the sliding manger knife handle 12 of the knife handle 1, and one end of said spring 4 is connected to the rear end of the knife seat 2, wherein another end of said spring 4 is connected with the rear end wall facet of the allocation portion of the knife handle 1.

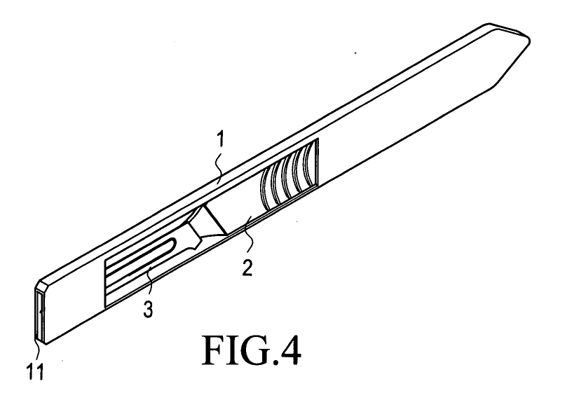

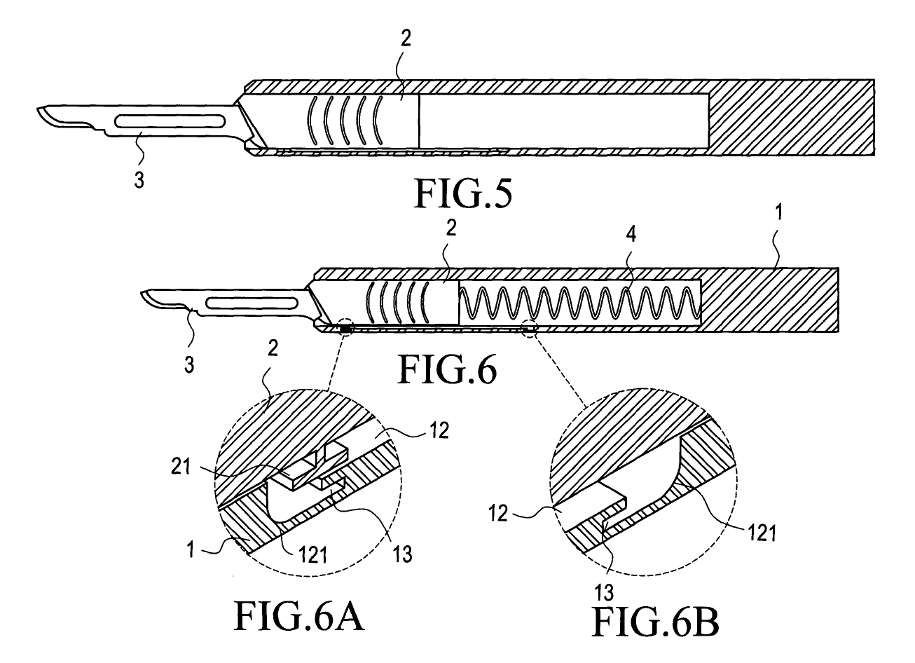

Please refer to FIG. 1, FIG. 2, FIG. 2A, FIG. 3, FIG. 4 as well as FIG. 5; the safety surgeon knife structure provided by this invention utilizes the spring force of the spring 4 to push forward the knife seat 2 such that the surgeon razor blade 3 on the front end of the knife seat 2 is penetrating out of the allocation portion knife handle 1; so the safety surgeon knife structure provided by this invention utilizes the sliding block 2 settled on the bottom of the knife seat 2 normally to settle within the manger 13 on the rear end of the sliding manger 12 of the knife handle 1; when using the safety surgeon knife structure provided by this invention, it only requires to push backwardly the knife seat 2 to make the sliding block 2 off the manger 13 on the rear end of the sliding manger 12, and guide the sliding block 2 entering into the sliding manger 12 through the guiding angle 12, next it pushes forward the knife seat 2 by utilizing the spring force of the spring 4 to make the razor blade 3 on the front end of the knife seat 2 penetrating out of the allocation portion knife handle 1, meanwhile, to push ahead the knife seat 2 continuously and to let the sliding block 2 on the knife seat 2 entering into the manger 13 on the front end of the sliding manger 12 by utilizing the guiding angle knife handle 12 on the front end of the sliding manger 12 so that the knife seat 2 is fixed on the front end of knife handle 1. After using the knife structure of this invention, it requires only to push ahead the knife seat 2 and guide the sliding block 2 knife handle 1 of the knife seat 2 onto the sliding manger 12 by utilizing the guiding angle 12 of the knife handle 1 on the rear end of the sliding manger, next to push backwardly to guide the sliding block 2 of the knife handle 1 on the rear end of the sliding manger 2 onto the manger 13, and to settle the sliding block 2 of the knife handle 1 on the manger by the elastic force of the spring 4 whereas the surgeon razor blade 3 is then allocated within the knife handle 1 to avoid the penetration of the razor blade 3 to cause dangerous; besides, it could also settle the pull force generated by the spring 4 to pull backwardly; also there is no spring for this invention.

Please refer to FIG. 6, FIG. 6A, FIG. 6B, during the structure of the safety surgeon knife structure provided by this invention the manger 13 settled on both ends of the sliding manger 12 of said knife handle 1 could also be settled on the bottom of the sliding manger 12, whereas the pull force generated by said spring 4 is to pull backwardly toward the knife seat 2 which makes the surgeon razor blade 3 be allocated within the allocation portion knife handle 1 wherein the sliding block 2 of the knife seat 2 is fixed within the manger 13 on the rear end of the sliding manger 12; when someone wants to use the safety surgeon knife structure provided by this invention, he only requires to pull back the knife seat 2 to make the sliding block 2 of the knife handle 1 lift-off the manger 13 on the rear end of the sliding manger 12, next to push the knife seat 2 upwardly to make the sliding block 2 entering into the sliding manger 12, next to push ahead the knife seat 2 so that the sliding block 2 is sliding ahead the front end of the sliding manger 12, meanwhile, it pushes downwardly and pull backwardly to buck the sliding block 2 of the knife seat 2 into the manger 13 on the front end of the sliding manger 12 which fixes the knife seat 2 on the front end of the knife handle 1 and penetrates the razor blade 3 outside the allocation portion knife handle 1 to proceed surgeon. After using the surgeon knife provided by this invention, it only requires to push the knife seat 2 ahead and next to push it upwardly to make the sliding block 2 entering into the sliding manger 12, next to pull the knife seat 2 onto the rear of the allocation manger of the knife handle 1 by utilizing the pull force of the spring 4. Next to fix the sliding block 2 of the knife handle 1 onto the manger 13 on the rear of the sliding manger 12 by pushing the knife seat 2 to avoid the penetration of the surgeon razor blade 3 to cause dangerous; besides, said spring 4 could also be set-up as the elastic force generated by the spring 4 to push ahead the knife seat 2.

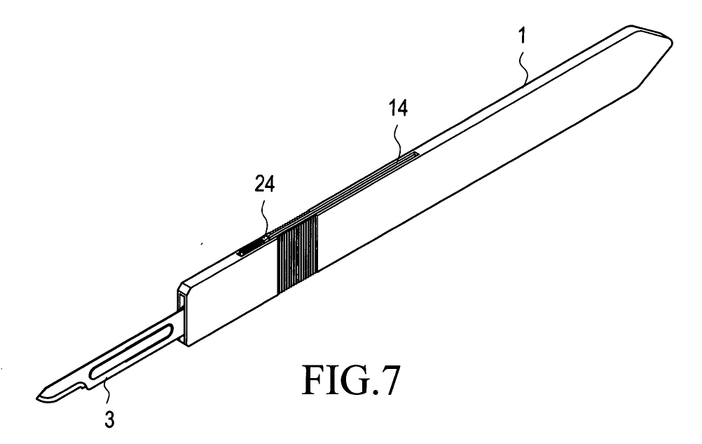

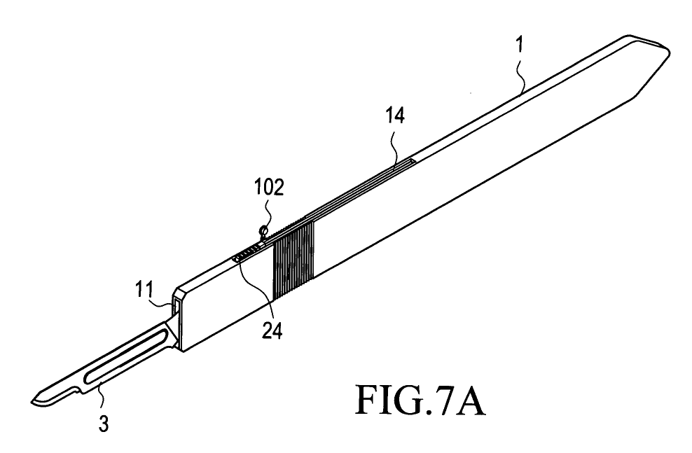

Please refer to FIG. 7 and FIG. 7A, the safety surgeon knife structure provided by this invention, one lateral of said knife handle 1 there settles a hole 14 in penetrating with the allocation portion knife handle 1 which also could be settled on the top of the knife handle 1, wherein there settles a holding portion 24 on the knife seat 2 which penetrates out of said hole 14, to control the position of the sliding block 2 of knife handle 1 settled by the knife seat 2 by means of initiating said holding portion 24 wherein the knife seat 2 is fixed on the front end or the rear end of the knife handle 1; besides, there settles a column plug knife handle 102 on said knife handle 1, when the holding portion 24 is moving sliding on the front end of the hole 14, the column plug 102 could be plugged into the hole 14 on the rear lateral of the holding portion 24 to avoid the sliding backwardly of the holding portion.

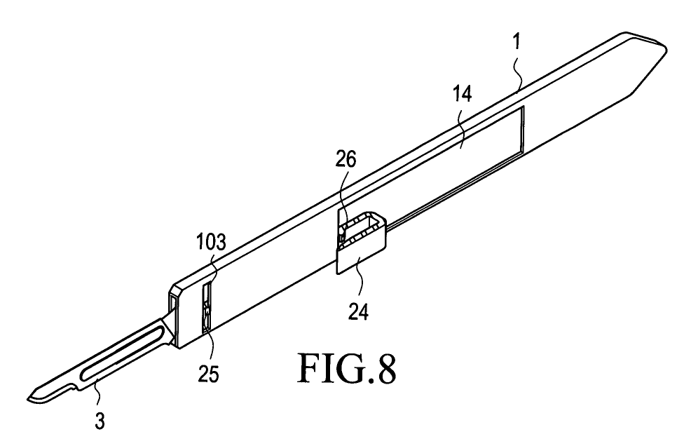

Please refer to FIG. 8, during the safety surgeon knife structure provided by this invention, there could settle a fixed hole 103 on the front end of the hole knife handle 14 of the knife handle 1; besides, there could also not to settle the sliding manger 12 and the manger 13 within the allocation portion of the knife handle 1, and there settles a holding portion on the rear end of the knife seat 2, and there settles a pre-buckle 25 which is pivot and fixed within the fixed hole 103, and there also settles a buck block 26 on the front end of the hole knife handle 14 settled on the rear end of the pre-buckle 25; by means of press-down the holding portion 24 and to pull backwardly, it could let the pre-buck 25 lift-off the fixed hole 103 and slide within the hole 14, wherein the front end of the pre-buck 25 there touches the wall of the front edge of the hole to make the razor blade 3 keeping within the allocation manger of the knife handle 1.

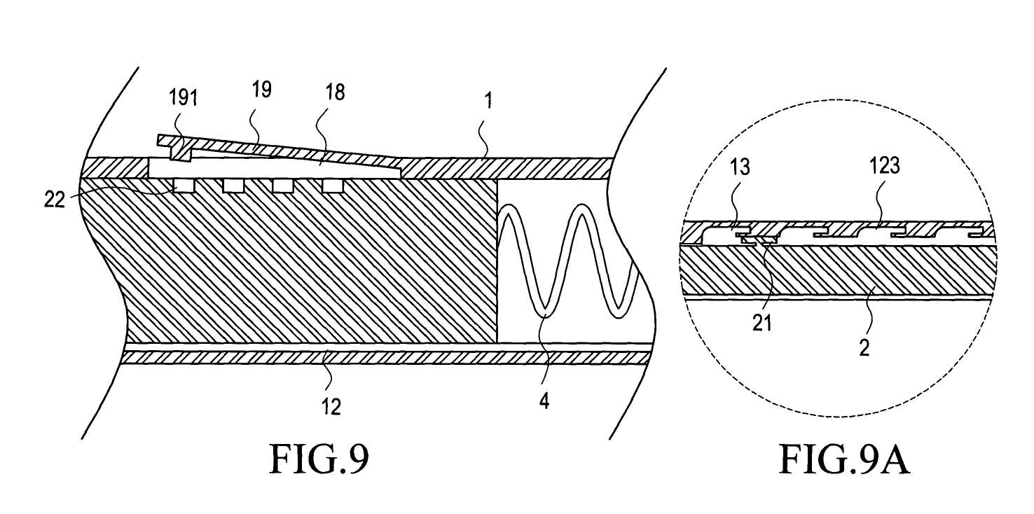

Please refer to FIG. 9 and FIG. 9A, during the safety structure of safety surgeon knife, there could settle a void 18 which penetrates through the wall facet of the knife handle 1 on the top or the bottom of said knife handle 1, and there settles a spring sheet 19 on the peripheral of said hole 18, and there places a convex buck 19 of the knife handle 1 on the end of said spring sheet 19, and there settles a plurality of buck holes 22 on the top or bottom facet of said knife seat 2; when the surgeon razor blade 3 penetrates out of the allocation portion knife handle 1, it could utilize the convex buck 19 of the knife handle 1 on the end of the spring sheet 19 to buckle within the buck hole 22 to avoid the sliding of the razor blade 3 generated on surgeon; besides, there could also make some scales on the top or bottom of said knife seat 2 which acquire the degree of buck hole 22 of the convex buck 19 of the knife handle 1 by the observation of the relative position of the convex buck 19 of the knife handle 1 and the scales to control the length of the razor blade 3 out of the allocation portion 3; next, there settles a sliding manger 12 on both ends of the sliding manger 12 which could not only settle the manger 13 on both ends of the sliding manger 12 but also settles a plurality of positioning manger 123 on the lateral or bottom of the sliding manger 12 to control the position of the surgeon razor blade 3 through buckling the sliding block 2 of the knife handle 1 into each positioning manger 123.

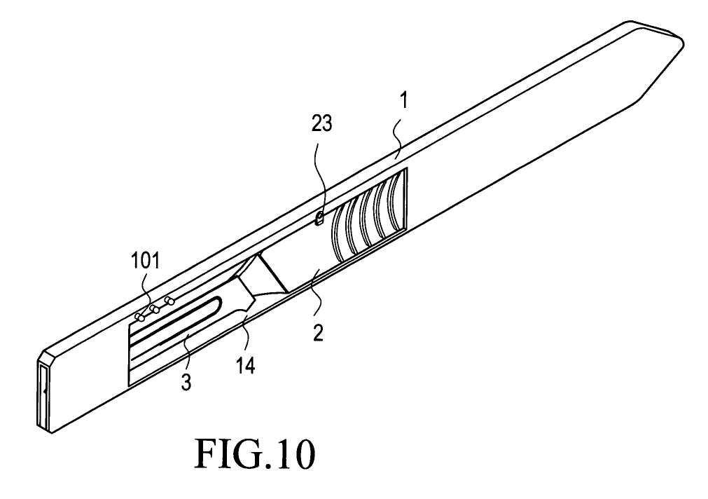

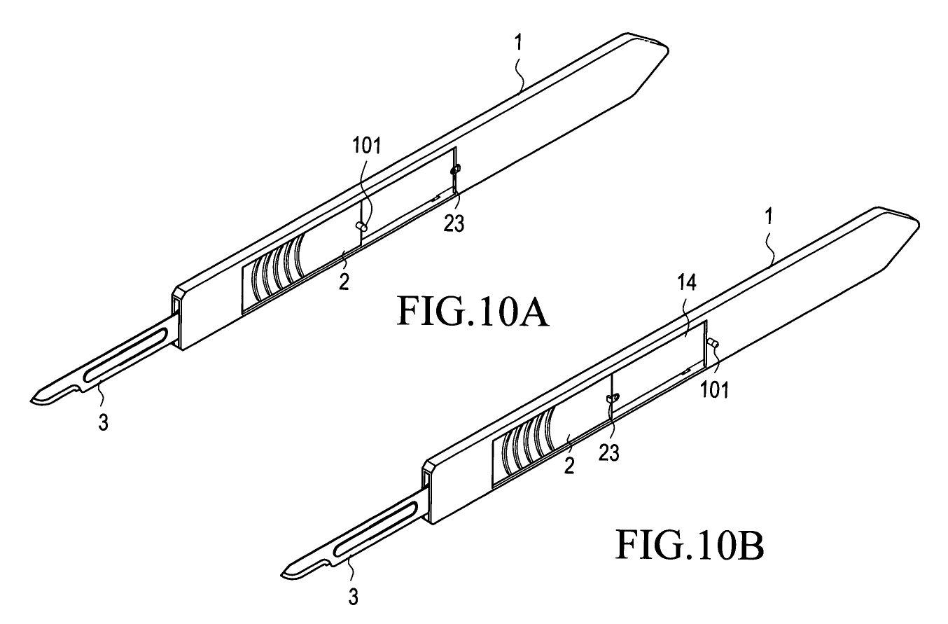

Please refer to FIG. 10, FIG. 10A & FIG. 10B, on the surgeon knife structure provided by this invention, there settles a plurality of buck 10 of the knife handle 1 on one lateral of said knife handle 1, and there settles a movable round buck 23 which penetrates out of the penetrating hole 14 of the knife handle 1 on said knife seat 2; when the knife seat 2 is sliding onto the front end of the knife handle 1, it could buckle on said buck 10 of the knife handle 1 through said movable round buck 23; besides, it fixes the knife seat 2 on the front end of the knife handle 1 which avoids the phenomena of sliding of the razor blade 3 on surgeon; also it controls the length of the razor blade 3 penetrating out of the allocation portion 3; besides, said buck 10 could also be settled on the rear end of the knife seat 2, whereas the movable round buck 23 is settled on the rear end of the hole of the knife handle 1; besides, said movable round buck 23 could be settled on the rear end of the hole of the knife handle 1 wherein the buck 10 is settled on the rear end of knife seat 2.

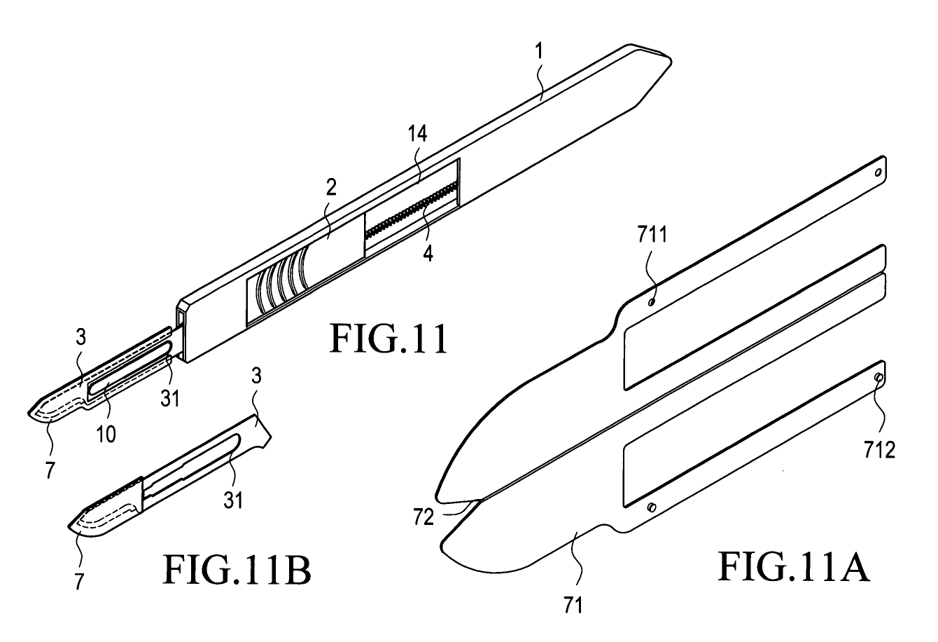

Please refer to FIG. 11, FIG. 11A & FIG. 11B, the safety surgeon knife structure provided by this invention, there could shelf settle a protecting envelop 7 on the outer edge of said surgeon to avoid the outer-exposure of the portion of said surgeon knife; wherein said protecting envelop 7 is compromised of two sheets body 7 of the knife handle 1 which is in connection utilizing a flexture portion 72 wherein there settles a plurality of buck hole 7 of the knife handle 1 on one sheet body and there also settles a buck block 12 on another sheet body in corresponding with the position of a buck hole 7 of knife handle 1; before using the safety surgeon knife provided by this invention, said protecting envelop is enveloped and settled on the surgeon razor blade 3; so when using it must pushes the surgeon razor blade 3 out of the allocation portion of the knife handle 1, next move one sheet body 7 of the knife handle 1 of the protecting envelop 7 to separate the buck block 712 with the buck hole 71 through the elastic force of the sheet body 71 to extend out the protecting envelop 7 and out-exposure the surgeon razor blade 3; besides, said protecting envelop 7 could also be made to form with-a-whole.

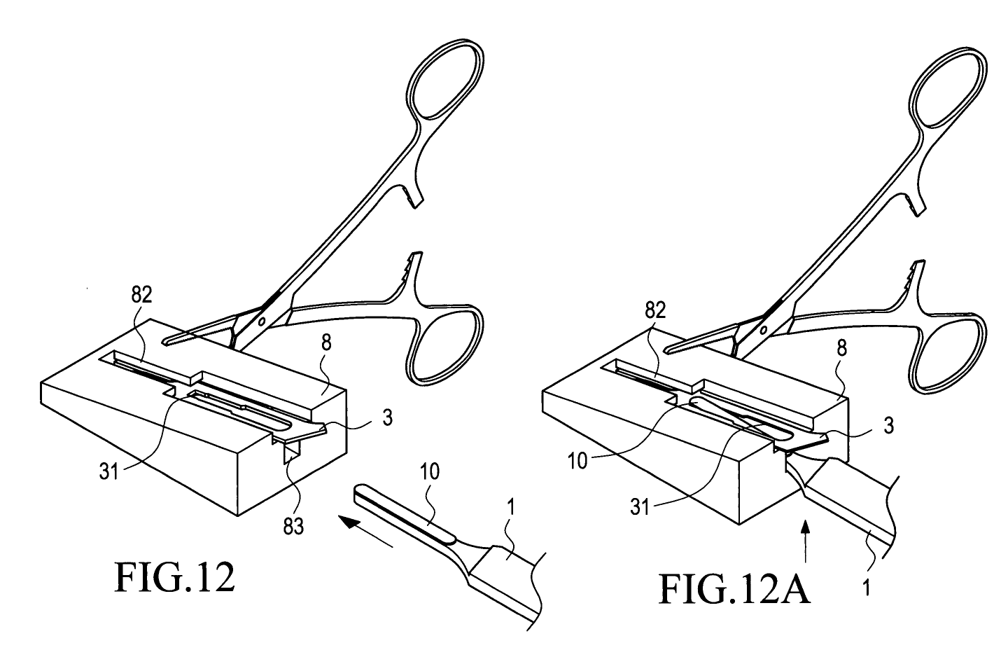

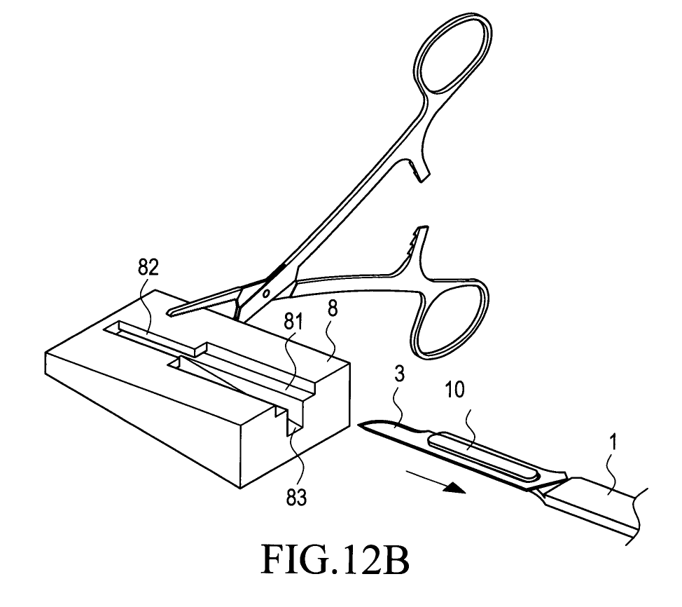

Please refer to FIG. 12, FIG. 12A & FIG. 12B, the safety surgeon knife structure provided by this invention having the knife seat 2 wherein it could not to settle the surgeon razor blade 3 on the front end of it, and there settles a holding seat 10 on the front end of the knife seat 2, wherein there settles a putting seat 8 on the surgeon razor blade 3, there form a an inclination plane on the top facet of said putting seat 8, there opens a knife-putting manger 8 of the knife handle 1 inserted for the surgeon razor blade 3, and there settles a convex block 82 which could cover the razor portion on the corresponding position on the front end of the knife-putting manger 8 knife handle 1, and there settles a concave manager 83 on the position of the hole 3 of the knife handle 1 on the bottom of the knife-putting manger 81 which corresponds to the position of the portion of the razor blade 3, and said concave manager 83 penetrates through the rear end of the putting seat 8, when using the safety surgeon knife structure provided by this invention, first to use a needle-holder 84 to clamp the putting seat 8, next to push forward the knife seat 2, which makes the holding seat 10 on the front end of the knife seat 2 penetrates out of the allocation portion of the knife handle 1, next to penetrate the holding seat 10 into the concave manger 83 of the putting seat 8; besides, since said knife-putting manger 81 is on the same incline facet with the top facet of putting seat 8, there will generate an angle between the holding seat 10 entering from the concave manger 83 and the surgeon razor blade 3, wherein the front end of the holding seat 10 will penetrate through the holding hole 31 of the razor blade 3, next to push upwardly the knife handle 1 or to push downwardly the needle-holder 84 of the putting seat 8, meanwhile, it fixed Razor blade 3 by the convex block 82 to fix the razor blade 3 by using the holding seat 10, next, it could proceed operation after taking off the razor blade 3 from the putting seat 8. Besides, when the surgeon is under processing or the surgeon is about to complete, if we want to take off the surgeon razor blade 3 from the knife handle 1, it only requires to insert the knife handle 1 in addition to the surgeon razor blade 3 into the concave manger 83 of the putting seat 8 and the knife putting manger 81, and to place them downwardly the knife handle 1 or to the needle-holder 84 of the putting seat 8 which let the rear portion of the holding seat 10 life-off the holding hole 31 of the razor blade 3, next to take off the surgeon razor blade 3 from the concave manger 83 of the putting seat 8, wherein the razor blade 3 is separated from the knife handle 1.

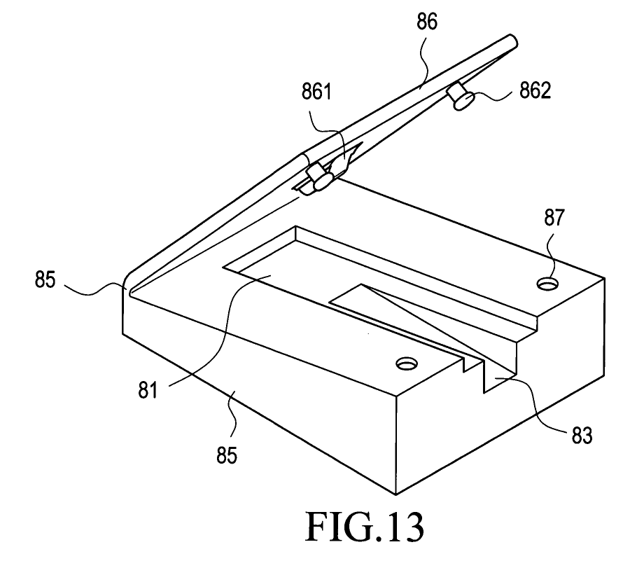

Please refer to FIG. 13, there connects an upper cap 86 with an elastic portion 85 on said putting seat 8, wherein there concave settles a portion 861 on the concave manger 83 position of the dynamic putting seat 8 on the middle bottom of said upper cap 86, and there settles a plurality of buck 87 on the top facet of said putting seat 8, and there settles a plurality of buck piece 862 on the position of the corresponding buck hole 87 on the bottom of said upper cap 86, when the surgeon razor blade 3 is put in the middle of the knife putting manger 8 of the knife handle 1, there uses the buck piece 862 to buck into the holding hole 87 of the putting seat 8 for said upper cap 86, meanwhile the portion 861 on the bottom of said upper cap 86 is within the holding hole 31 of the razor blade 3; so when the holding seat 10 is penetrating into the concave manger 83, the holding seat 10 will touch with the razor blade hole 3 of the razor blade, and to make it open the buck piece 862 of the upper cap 86 and the buck hole 87 of the putting seat 8 so that the holding seat 10 could be taken away from the putting seat 8 along with the 3; and when the holding seat 10 is being taken away from the putting seat 10 and separates with the razor blade 3, the buck piece 862 of the upper cap 86 is embedded with the buck hole 87 of the putting seat 8 by means of the holding force of the needle holder 84 clamping on the putting seat 8 to fix the razor blade 3 onto the putting seat 8; besides, the above-mentioned method could also be used in the conventional surgeon knife.

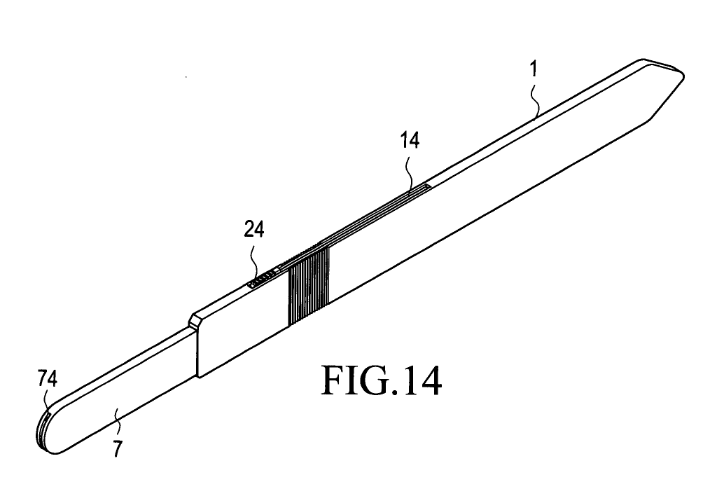

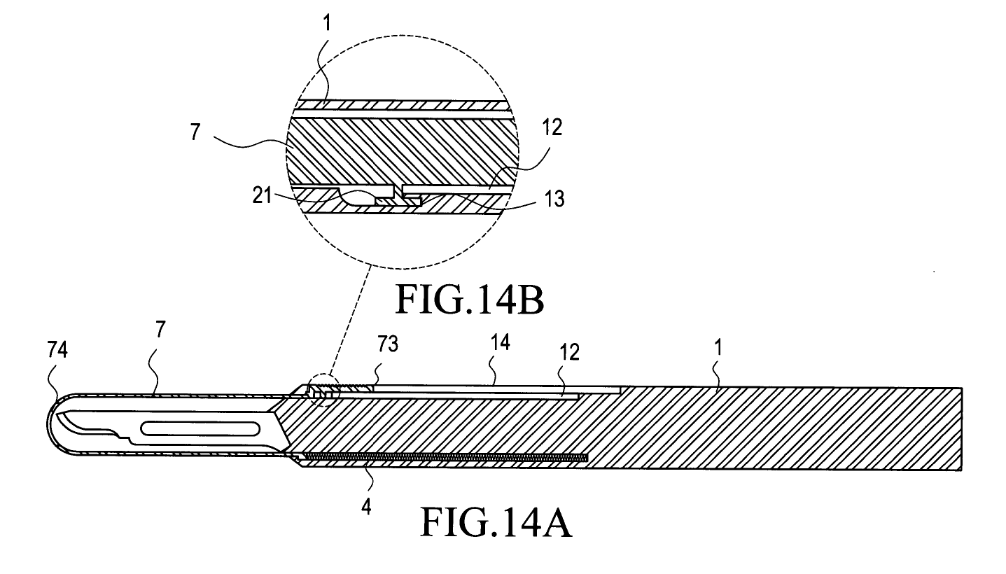

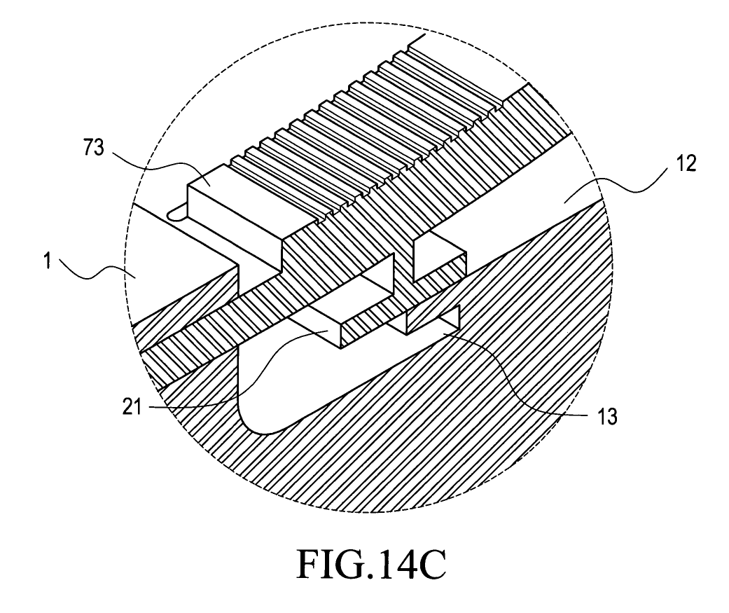

Please refer to FIG. 14, FIG. 14A, FIG. 14B as well as FIG. 14C, the safety surgeon knife structure provided by this invention comprises mainly:
A knife handle 1, on the front end of said knife handle there settles a surgeon razor blade 3, and there enclosed settles a sliding manger 12 on the inner portion of the front end of the knife handle 1, and there settles a hole 14 on the top facet of said knife handle 1 in connection with the sliding manger 12 on the top facet, and there both settles a manger 13 in parallel with the sliding manger 12 on both ends of the bottom of the top facet;
A protective coat 7, there settles an opening 74 on the front end of said knife handle 1, and also settles a push-press sheet 73 on the rear end of said protective coat 7, and there settles a sliding block 21 on the bottom of the front end of said push-press sheet 73; and
A spring 4, one end of said spring 4 is connected with the rear end of the protective coat 7, wherein another end of said spring 4 is connected to the wall facet of the rear end of the sliding manger 12 in connection with the knife handle 1.

Said protective coat 7 is penetrated within the sliding manger knife handle 12 of the knife handle 1, and the push-press sheet 73 on said protective coat 7 is penetrated from the hole 14 on the top of the knife handle 1, wherein the sliding block 21 of the push-press sheet 73 is put within the sliding manger 12 of the knife handle 1; the safety surgeon knife structure provided by this invention the protective coat 7 with the elastic force of the spring 4, which enclosed and enveloped the surgeon razor blade 3 on the front end of the knife handle 1 from the protective coat 7, and the sliding block 21 of the protective coat 7 is settled within the manger 13 on the front end of the sliding manger 12 of the knife handle 1; when using the safety surgeon knife structure provided by this invention, it must push forward the push-press sheet 73 of the protective coat 7 so that the sliding block 21 beneath the push-press sheet 73 lift-off the manger 13 on the front end of manger 13, next to push the push-press sheet 73 from the lateral so that the push-press sheet 73 moves forward the sliding block 21 and enter into the sliding manger 12 and pull backwardly the push-press sheet 73 wherein the protective coat 7 is back to the rear of the knife handle 1; meanwhile, push the push-press sheet 73 to the side first and move it ahead so that the sliding block 21 on the beneath of the push-press sheet is buckled within the manger 13 on the backward of the knife handle 1 such that the protective coat 7 is fixed behind the knife handle 1, and then the surgeon could be proceeded by using the surgeon knife penetrating out of the opening 74 of the protective coat 7; besides, there could also settle the manger 13 on both ends of said knife handle 1; before using the safety surgeon knife structure provided by this invention, the sliding block 21 of the protective coat 7 is settled and buckled within the manger 13 on the front end of the sliding manger 12 of the knife handle 1, so there will have a little up-righting for the rear end of said push-press sheet 73, when using the safety surgeon knife structure of this invention, it must first push forward the push-press sheet 73 of the protective coat 7, and press down the rear end of the push-press sheet 73 so that the sliding block 21 entering into the track of the sliding manger 12, next to press down the push-press sheet 73 and pull it down backwardly through the rear end of the sliding manger, next to press down the front end of the push-press sheet 73 and pushes it forwardly so that the blocking block 21 on the bottom of the push-press sheet 73 entering into the manger 13 on the rear end of the sliding manger 12 which makes the protective coat 7 being fixed on the rear end of the knife handle 1, and the surgeon razor blade 3 on the front end of the knife handle 1 is penetrating out from the opening 74 on the front end of the protective coat 7; besides, there could also not to settle the spring 4 to be in connection with the wall facet on the rear end of the allocation manger knife handle 1; also there could not to settle the push-press sheet 73 on the rear end of the protective coat 7, and the sliding block 21 is settled on the bottom or the inner of the protective coat 7 wherein the penetrating hole of said knife handle 1 is settled on the lateral of the knife handle 1.

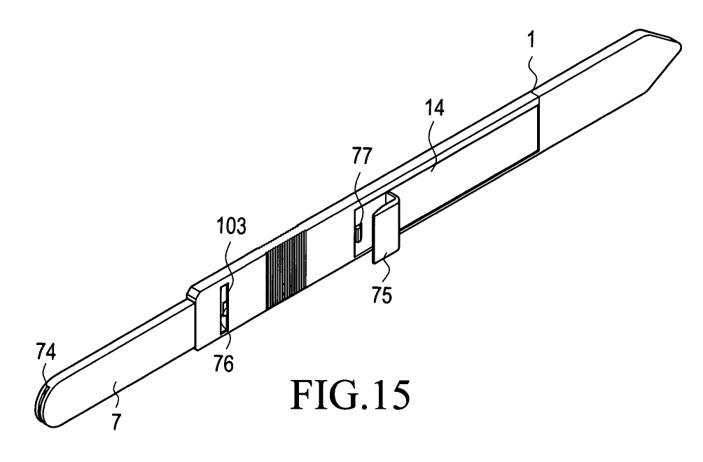

Please refer to FIG. 15, among the safety surgeon knife structure provided by this invention, there could settle a fixed hole 103 on the front end of the hole 14 of said knife handle 1, wherein there settles a push-moving sheet 75 on the rear end of the protective coat 7, and there settles a pre-buckle 76 which is buckled and settled within the fixed hole 103, and there settles a buck bump 77 on the front end of the hole 74 settled on the rear end of the pre-buckle 76, the pre-buckle 76 could be made to life-off the fixed hole 103 and sliding within the hole 74 by press-down the push-and-moving sheet 7 and next to pull it back, and the front end of the pre-buckle 76 touches the front edge wall facet of the hole 14 which makes the razor blade 3 penetrating out from the opening 74 on the front end of the protective coat 7.

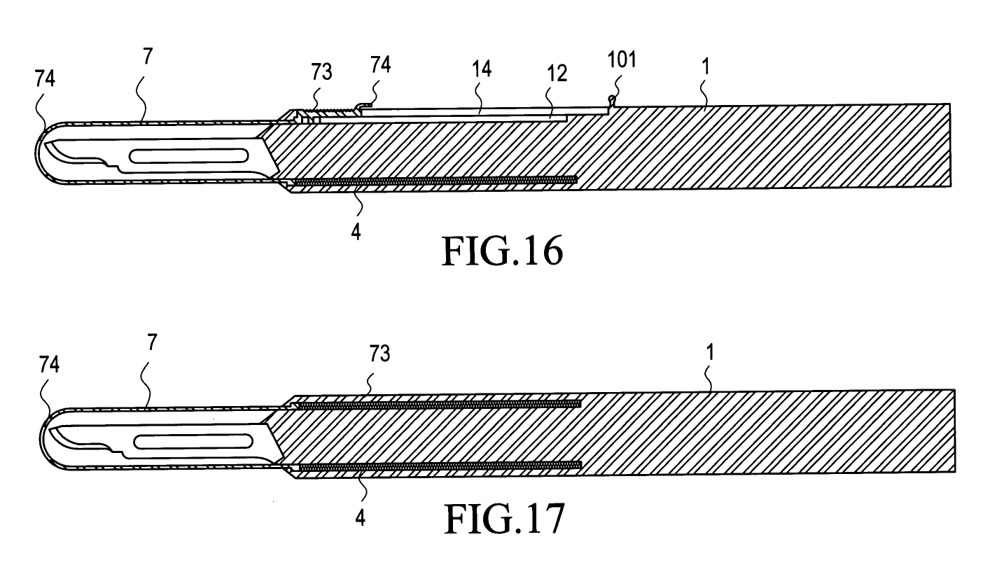

Please refer to FIG. 16, among the safety surgeon knife provided by this invention, there settles a buck spring 74 on the rear end of the press sheet 73 of said protective coat 7, and there settles a buckle knife handle 101 on the rear end of the hole knife handle 14 of said knife handle 1, so when the press sheet 73 is sliding onto the rear lateral of the hole 14, the protective coat 7 could be fixed on the rear end of the knife handle 1 by bucking said buck ring 74 onto the buckle knife handle 101 which out-penetrating the razor blade 3 to proceed the operation and avoid the translocation of the protective coat 7 on surgeon to stop the surgeon; besides, there could also settle the buck ring 74 on the rear end of the hole 14 of the knife handle 1 wherein the buckle knife handle 101 is settled on the rear end of the push-press sheet 73.

Please refer to FIG. 17, among the safety surgeon knife structure provided by this invention, there enclosed settles a sliding manger 12 on the inner portion of the front end of said knife handle 1, and the protective coat 7 is settled within the sliding manger 12 of the knife handle 1, there settles a spring 4 on the rear end of the protective coat 7 which is in connection with the rear end wall facet of the sliding manger 12, and said protective coat is made up of a transparent substance, before using the safety surgeon knife provided by this invention, the spring 4 on the rear end of the protective coat 7 utilizes the elastic force to push forward the protective coat 7 onto the front end of the knife handle 1, so when using the safety surgeon knife structure provided by this invention, it only requires to touch the protective coat 7 onto the operation portion and next to apply forces to compromise the elastic force of the spring 4 to pull the protective coat 7 backwardly to make the surgeon razor blade 3 penetrating out to proceed the operation; besides, since the protective coat 7 is made up of the transparent substance, the cutting depth of the razor blade 3 could thus be acquired through the protective coat 7; and after finish the safety surgeon knife structure of this invention, it only requires to move apart the safety surgeon knife structure of this invention from the operation portion to avoid the danger caused by the penetration of the razor blade 3.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

1. a safety surgeon knife structure comprising:
a knife handle, there concavely settles an allocation portion from the front end, there settles a sliding manger on the bottom of said allocation portion, a reversely hook manger in parallel with the sliding manger, and there settles a plurality of holes in connection with the allocation portion;
a knife seat, wherein there settles within the allocation portion of the knife handle, and there settles a sliding block putting insert within the sliding manger of the knife handle; and
a spring, one end of said spring is connected on the rear end of the knife seat whereas another end is in connection with the rear end wall facet of the knife handle allocation portion.

2. the safety surgeon knife structure as mentioned in claim 1, wherein there could also settles the holes on the top of the knife handle wherein one wrench and hold portion penetrating out of said holes.

3. the safety surgeon knife structure as mentioned in claim 1, wherein there could settle a cylinder plug, when the wrench and hold portion is sliding onto the front end of the holes, the cylinder plug could be plugged insert the hole on the rear lateral of the wrench and hold portion to avoid the sliding and moving of the wrench and hold portion.

4. the safety surgeon knife structure as mentioned in claim 1, wherein there could also not to settle the spring for the safety surgeon knife structure.

5. the safety surgeon knife structure as mentioned in claim 1, wherein the reversely hook manger could also be concavely settled on the bottom of the sliding manger.

6. the safety surgeon knife structure as mentioned in claim 1, wherein there settles a fixed hole on the front of the holes of said knife handle, and there could also not to settle the sliding manger and the reversely hook manger on the allocation portion of the knife handle, and there settles a wrench and hold portion on the rear end of the knife seat; and there settles a buck block on the rear end of the pre-buckle.

7. the safety surgeon knife structure as mentioned in claim 1, wherein there could also settle a hole penetrating onto the wall facet of the knife handle on the top or the bottom of said knife handle, and there settles a spring on its peripheral of said hole, and there settles a convex buck, and there settles a plurality of bucks on the top or the bottom of said knife seat.

8. the safety surgeon knife structure as mentioned in claim 1, wherein there could also settles a plurality of positioning manger on the lateral or bottom of the sliding manger except for setting up the reversely hook manger on the front and rear end of the sliding manger.

9. the safety surgeon knife structure as mentioned in claim 1, wherein there settles a plurality of hook on the peripheral edge of said knife handle.

10. the safety surgeon knife structure as mentioned in claim 1, wherein there settles a buck ring on the rear end of said push-press sheet and there settles a hook on the rear end of the hole of said knife handle.

11. a safety surgeon knife structure comprising:
a knife handle, there concavely settles an allocation portion from the front end, there settles a sliding manger on the bottom of said allocation portion, a reversely hook manger in parallel with the sliding manger, and there settles a plurality of holes in connection with the allocation portion;
a protective coat, wherein there settles a push-and-press sheet on said protective coat, and there settles a sliding block putting insert within the sliding manger of the knife handle; and
a spring, one end of said spring is connected on the rear end of the knife seat whereas another end is in connection with the rear end wall facet of the knife handle allocation portion.

12. the safety surgeon knife structure as mentioned in claim 11, wherein there could also settles the holes on the top of the knife handle wherein one wrench and hold portion penetrating out of said holes.

13. the safety surgeon knife structure as mentioned in claim 11, wherein there could settle a cylinder plug, when the wrench and hold portion is sliding onto the front end of the holes, the cylinder plug could be plugged insert the hole on the rear lateral of the wrench and hold portion to avoid the sliding and moving of the wrench and hold portion.

14. the safety surgeon knife structure as mentioned in claim 11, wherein there could also not to settle the spring for the safety surgeon knife structure.

15. the safety surgeon knife structure as mentioned in claim 11, wherein the reversely hook manger could also be concavely settled on the bottom of the sliding manger.

16. the safety surgeon knife structure as mentioned in claim 11, wherein there settles a fixed hole on the front of the holes of said knife handle, and there could also not to settle the sliding manger and the reversely hook manger on the allocation portion of the knife handle, and there settles a wrench and hold portion on the rear end of the knife seat; and there settles a buck block on the rear end of the pre-buckle.

17. the safety surgeon knife structure as mentioned in claim 11, wherein there could also settle a hole penetrating onto the wall facet of the knife handle on the top or the bottom of said knife handle, and there settles a spring on its peripheral of said hole, and there settles a convex buck, and there settles a plurality of bucks on the top or the bottom of said knife seat.

18. the safety surgeon knife structure as mentioned in claim 11, wherein there could also settles a plurality of positioning manger on the lateral or bottom of the sliding manger except for setting up the reversely hook manger on the front and rear end of the sliding manger.

19. the safety surgeon knife structure as mentioned in claim 11, wherein there settles a plurality of hook on the peripheral edge of said knife handle.

20. the safety surgeon knife structure as mentioned in claim 11, wherein there settles a buck ring on the rear end of said push-press sheet and there settles a hook on the rear end of the hole of said knife handle.

21. the safety surgeon knife structure as mentioned in claim 11, wherein there settles a fixed hole on the front end of said knife handle, and there settles a moving and sliding sheet on the rear end of the protective coat, and there settles a pre-hook hooked within the fixed hole, and there settles a buck block touched on the front end of the hole.

22. a safety surgeon knife structure comprising:
a knife handle, there concavely settles an allocation portion from the front end, there settles a sliding manger on the bottom of said allocation portion, a reversely hook manger;
a surgeon razor blade, there settles a touch hole;
a protective coat, wherein there settles a push-and-press sheet on said protective coat, and there settles a sliding block putting insert within the sliding manger of the knife handle.

23. the safety surgeon knife structure as mentioned in claim 22, wherein there could also be formed in-a-while for said protective coat.

24. the safety surgeon knife structure as mentioned in claim 22, wherein there could be replaced with a putting seat for said protective coat, and there forms an inclination facet on the top of said putting seat, and there settles a knife-purring manger for the insertion of the surgeon knife, and there opens a concave manger on the position of the touch hole of the edge of the knife; and there settles a concave manger on the bottom of said knife putting manger, and said concave manger is penetrated the rear end of the putting seat.

25. the safety surgeon knife structure as mentioned in claim 24, wherein there could elastically connected with an upper cap on said putting seat, there convexly settles a touch portion on the dynamically putting position of the bottom of said upper cap, and there settles a plurality of buck holes and there settles a plurality of buck pieces on the corresponding positions on the bottom of said upper cap.
